(19) United States
(12) Patent Application Publication
(10) Pub. No.: US 2010/0049268 A1
(43) Pub. Date: Feb. 25, 2010

(54) MASTER/SLAVE PROCESSOR CONFIGURATION WITH FAULT RECOVERY

(75) Inventor: Antonio Garcia Martins, East Elmhurst, NY (US)

Correspondence Address: THORNE & HALAJIAN;APPLIED TECHNOLOGY CENTER, 111 WEST MAIN STREET, BAY SHORE, NY, 11706, US

(73) Assignee: AVERY BIOMEDICAL DEVICES, INC., Commack, NY (US)

(21) Appl. No.: 12/528,001

(22) PCT Filed: Feb. 20, 2008
(86) PCT No.: PCT/US08/54467

§ 371 Date: Aug. 20, 2009

Related U.S. Application Data
(60) Provisional application No. 60/890,633, filed on Feb. 20, 2007.

(51) Int. Cl. A61N 1/362; G06F 11/20

(52) U.S. Cl. 607/9; 714/11; 714/E11.072

(57) ABSTRACT

A fault-tolerant processor device including a master processor and a plurality of operationally coupled slave processors. The master processor sends a command to each of the slave processors to initiate operation to each control a different one of a plurality of operations during fault-free operation. The master processor monitors each of the operations to confirm the fault-free operation. In a case wherein fault-free operation is not confirmed, the master processor identifies a faulty one of the slave processors, disables the faulty slave processor and initiates operation of a fault-free one of the slave processors to control the operations of the faulty slave processor in addition to the operations of the fault-free slave processor. If the master processor determines that both of the slave processors are faulty, the master processor may disable both of the slave processors and control each of the operations independent of the faulty slave processors.

This application claims the benefit of U.S. Provisional Patent Application No. 60/890,633, filed Feb. 20, 2007.

The present system relates to a master/slave processor configuration with fault recovery having graceful degradation.

Fault-tolerant processing systems are known for systems wherein propagation of a processing fault is not acceptable. For example, in vehicle braking systems, fault intolerance is unacceptable and could lead to severe injury and property damage. System critical processes may have a built-in redundant system that may be hot-swappable to enable continued operation in the face of process failure. Redundant processor systems have two or more processors present to enable detection of a fault. For example, a lockstep processing system utilizes two processors, a master and slave, that each execute the same instructions utilizing a common system clock. Outputs from each processor are compared, either by an external comparing circuit or by one of the processors, to determine if the output of each of the processors is equivalent. In a case wherein the output is determined to be equivalent for the master and the slave, the processors are deemed to be fault-free and operation continues, typically with the master processor producing output results that are utilized by a downstream system. The slave processor output typically is only utilized by the compare circuit or by the slave itself for comparison to the master output. In a case wherein the master and slave outputs are not equivalent, the lockstep processor is deemed faulty and each of the master and slave processors may be disabled to avoid propagating an erroneous result to the downstream system.

In a fault-tolerant lockstep system, when the outputs of the master and slave are deemed not equivalent, a further test may be performed to determine which of the master or slave is at fault. In a case wherein the slave is determined to be faulty, the lockstep operation of the processors may be disabled and the master may continue to produce the output that is received by the downstream system. In a case wherein the master is determined to be faulty, the lockstep operation of the processors may be disabled and the slave may replace the master as the source of output for the downstream system.

In a further lockstep system, an idle processor being neither the master nor slave processor may be available to the system. In a case of a fault being determined to be present in either of the master or slave processor, the idle processor may be utilized to replace the faulty processor and thereby, continue lockstep operation.

In each of these systems, the output of only one of the processors is utilized for driving the downstream system with the second processor only acting as a piece of a fault detection system and in some case, as a backup processor.

It is an object of the present system to overcome disadvantages and/or make improvements in the prior art.

A fault-tolerant processor device including a master processor and a plurality of slave processors operationally coupled to the master processor. In operation, the master processor sends a command to each of the slave processors to initiate operation by the slave processors to each control a different one of a plurality of operations during fault-free operation. In one embodiment in accordance with the present system, the master processor monitors each of the operations to confirm the fault-free operation. In a case wherein fault-free operation is not confirmed, the master processor identifies a faulty one of the slave processors, disables the faulty slave processor and initiates operation of a fault-free one of the slave processors to control the operations of the faulty slave processor in addition to the operations of the fault-free slave processor.

In one embodiment, the master processor determines if both of the slave processors are faulty and if so, the master processor disables both of the slave processors and controls each of the operations independent of the faulty slave processors. The device may include a user input/output device operationally coupled to the master processor to produce a failure indication if one of the slave processors is faulty.

The slave processors may determine if the master processor sends the initiation command. In this embodiment, in a case wherein the master processor does not send the initiation command, for example within a predetermined period of time, the slave processors disable the master processor and each control the different one of the plurality of operations without the master processor initiating operation. In one embodiment, each of the plurality of slave processors monitors the operations of each other of the plurality of slave processors if the master processor is disabled. If the master processor is faulty, one or more of the slave processors may produce a failure indication.

During fault-free operation, the slave processors may acknowledge receipt of the initiation command to the master processor. The master processor may examine a timing of each of the operations to determine if there is fault-free operation. The device may be a diaphragmatic pacemaker, wherein each of the slave processors drives a different side of the diaphragmatic pacemaker. In one embodiment, a redundant power source may power one or more portions of the system.

It should be expressly understood that the drawings are included for illustrative purposes and do not represent the scope of the present system in which:

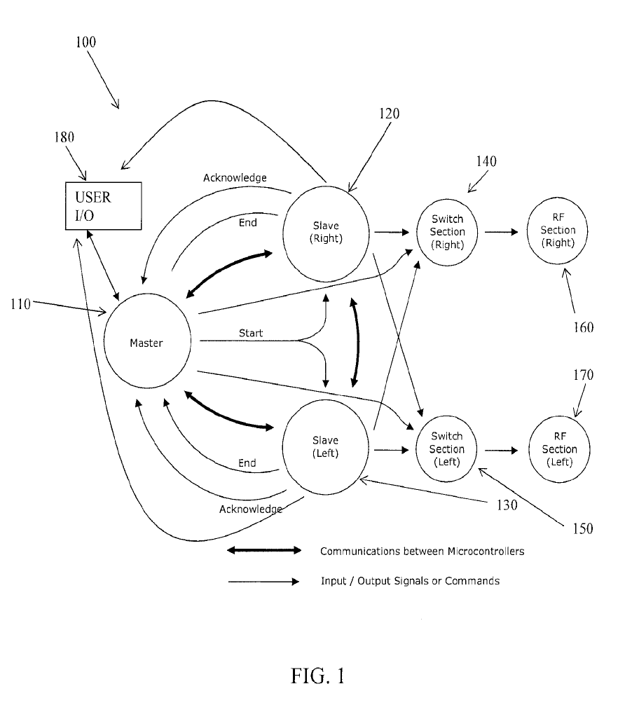

FIG. 1 shows an illustrative system in accordance with an embodiment of the present system;

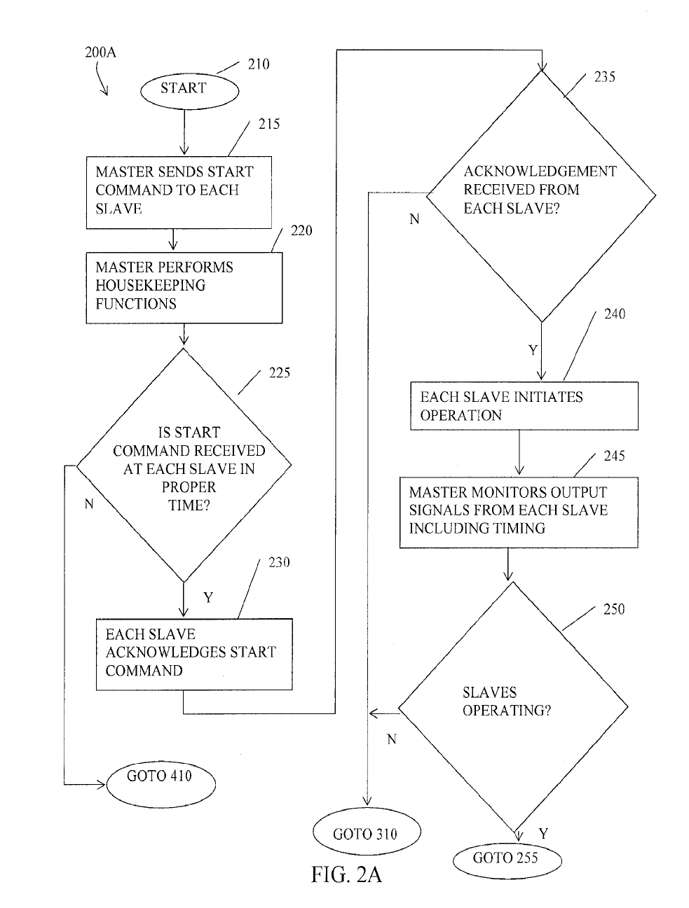

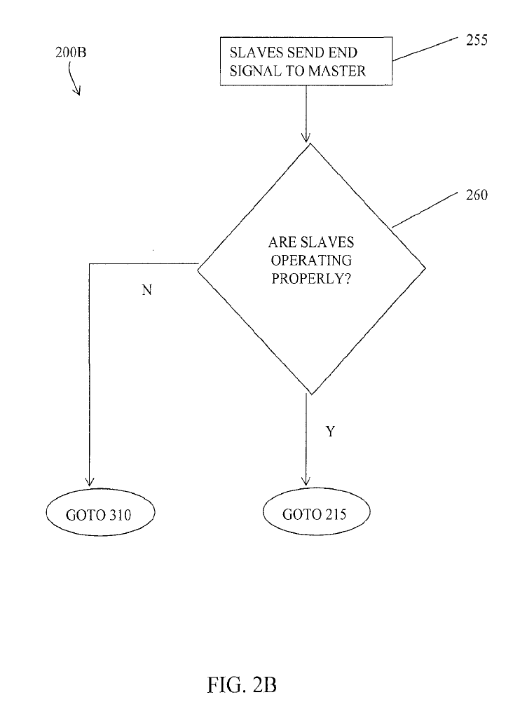

FIGS. 2A, 2B show a flow diagram illustrating failure-free operation in accordance with an embodiment of the present system;

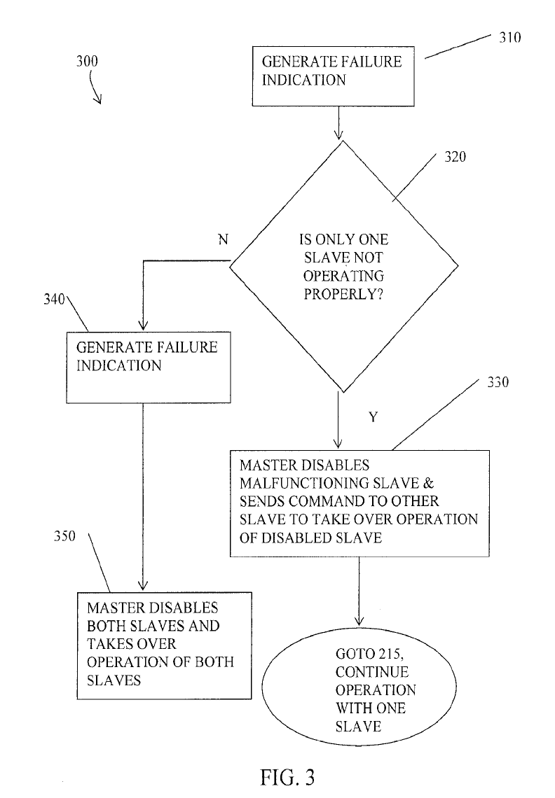

FIG. 3 shows a flow diagram illustrating a faulty slave processor operation in accordance with an embodiment of the present system;

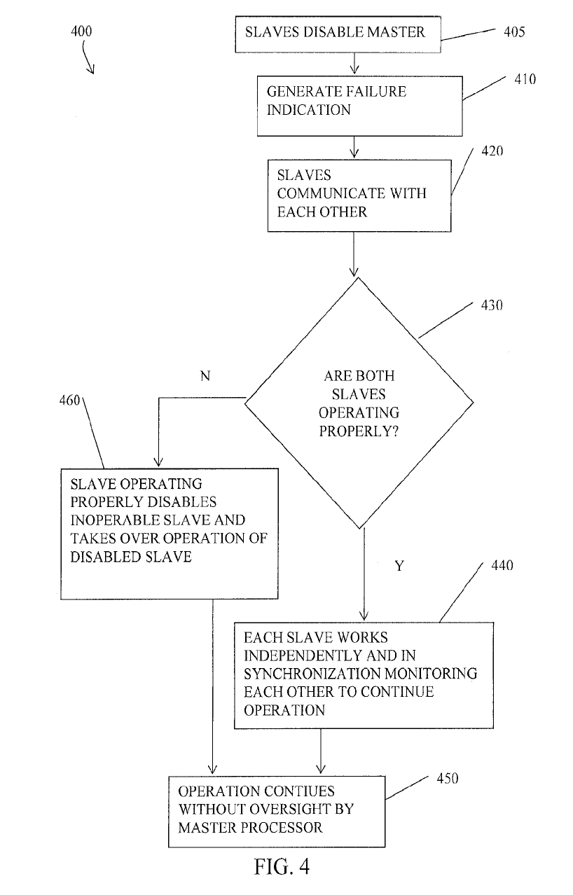

FIG. 4 shows a flow diagram illustrating a faulty master processor operation in accordance with an embodiment of the present system; and

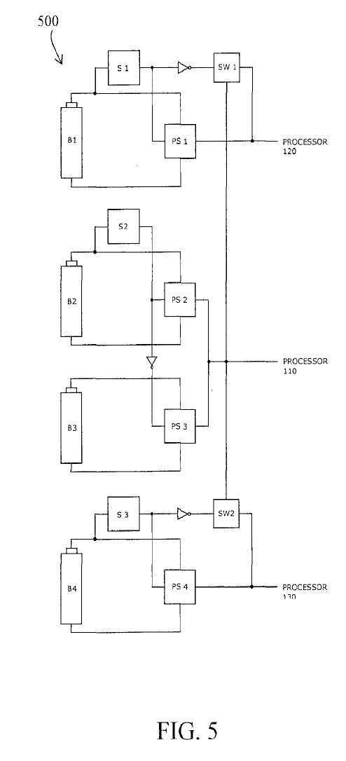

FIG. 5 shows a redundant power supply arrangement in accordance with an embodiment of the present system.

The following are descriptions of illustrative embodiments that when taken in conjunction with the following drawings will demonstrate the above noted features and advantages, as well as further ones. In the following description, for purposes of explanation rather than limitation, specific details are set forth such as architecture, interfaces, techniques, etc., for illustration. However, it will be apparent to those of ordinary skill in the art that other embodiments that depart from these details would still be understood to be within the scope of the appended claims. Moreover, for the purpose of clarity, detailed descriptions of well-known devices, circuits, and methods are omitted so as not to obscure the description of the present system.

FIG. 1 shows an illustrative system 100 in accordance with an embodiment of the present system. The system 100 includes a master processor 110, slave processors 120, 130 and switches 140, 150, each operationally coupled together to enable redundant failure operation as described further herein. The term “operationally coupled”, “coupled” and formatives thereof as utilized herein refer to a connection between devices and/or portions thereof that enables operation in accordance with the present system. The coupling may be wired, wireless, optical, and/or any other system that enables the operation. In a wireless coupling, the coupling may be radio-based (e.g., RF, Bluetooth, WiFi, etc.), infrared, optical, etc. The master processor 110 is also operationally coupled to a user input/output (I/O) device 180. The switch 140 is operationally coupled to an output system, illustratively shown as a Radio Frequency (RF) output section 160. The switch 150 is coupled to an output system, illustratively shown as an RF output section 170.

In one embodiment, the RF output sections may each output an amplitude-modulated or pulse width-modulated RF pulse train that may be received by an RF receiver for driving a diaphragmatic pacemaker. In other words, each of the switches 140, 150 may be utilized for driving a corresponding side of the diaphragmatic pacemaker. The switch 140 may be utilized to drive a right side of the diaphragmatic pacemaker while the switch 150 may be utilized to drive a left side of the diaphragmatic pacemaker (or vice a versa). As described above, each of the switches 140, 150 may be coupled to each of the processors 110, 120, 130 although in operation are only typically driven by one of the processors 110, 120, 130. Naturally, in other embodiments, the outputs of the switches 140, 150 may be utilized for driving other systems that may lend operation to being driven by one or more processors, such as for safety and/or other inherent fault intolerant systems. For example, in another embodiment, the processors 120, 130 may be respectively utilized for driving corresponding left and right portions of an antilock braking system (ABS). Other applications of the present system would readily occur to a person of ordinary skill in the art and are intended to be encompassed by the present system.

As is known in the art, the methods and apparatus discussed herein may be distributed as an article of manufacture that itself comprises a memory, such as integrated into one or more of the processors 110, 120, 130 or separated therefrom, having computer-readable code embodied thereon. The computer-readable code may be operable, in conjunction with the processors 110, 120, 130 to carry out all or some of the acts to perform the methods or create the apparatus discussed herein. The memory may be a recordable medium (e.g., floppy disks, hard drives, DVD, solid state memory, memory cards, etc.) or may be a transmission medium. Any medium known or developed that can store and/or provide information suitable for use with the system 100 may be used.

In one embodiment, the memory configures the processors 110, 120, 130 to implement the methods, acts, and/or functions disclosed herein. The memory may be implemented as electrical, magnetic or optical memory, or any combination of these or other types of storage devices. Moreover, the term “memory” should be construed broadly enough to encompass any information able to be read from or written to an address in an addressable space accessible by one or more of the processors 110, 120, 130. With this definition, information on a network is still within the memory since one or more of the processors 110, 120, 130 may retrieve/write the information from/to the network. It should also be noted that some or all of operations described herein may be incorporated into an application-specific or general-use integrated circuit including the operation of one or more of the processors 110, 120, 130 and the memory.

Further, one or more of the processors 110, 120, 130 may be dedicated processors for performing in accordance with the present system or may be general-purpose processors wherein only one of many functions operates for performing in accordance with the present system. The processors 110, 120, 130 may operate utilizing a program portion, multiple program segments, or may be a hardware device utilizing a dedicated or multi-purpose integrated circuit. For example, in one embodiment, one or more of the processors may be microcontrollers wherein operation in accordance with the present system may be embedded into the microcontroller directly, such as embedded memory, input/outputs, etc. In another embodiment wherein one or more of the processors 110, 120, 130 are microcontrollers, operation in accordance with the present system may be wholely or partly provided utilizing hardware and/or software programming.

The master processor 110 is operationally coupled to the user I/O 180 to facilitate operation within a user interface that may be provided through the user I/O 180. The coupling may be wired, wireless, and/or optical. For example, in one embodiment the user I/O 180 may include an infrared interface (IrDA) to communicate with a laptop computer allowing, for example, a review of operating parameters, a change of operating parameters, and/or downloading/uploading data, such as diagnostic data as described further herein. In general, the user I/O 180 may be utilized for interaction, including user interaction and/or interaction of another device, with the present system.

For example, the user I/O 180 may include an expansion I/O port that may allow the system 100 to be connected to external devices. In a diaphragmatic pacemaker application, the master processor 110 may, through use of the expansion I/O (e.g., the user I/O 180), be enabled to read an output signal from a pulse oximeter indicative of a user's oxygen saturation level. In operation, the master processor 110 may in response adjust a breathing rate and/or other parameters based on the user's oxygen saturation level.

In another embodiment, the present system 100, through use of the expansion I/O, may receive a signal or signals from a device that amplifies, decodes, and/or transmits signals originating from another source system, such as signals originating from the user's brain, nerves and/or other electrical systems (e.g., internal electrical system, external electrical system, etc.), to trigger the present system 100, such as triggering the present system 100 to initiate diaphragm extension.

In accordance with the present system, the system 100 through use of the expansion I/O may respond and/or sense any one or more of any physiological signal including voltage signals from the brain, nerves, and/or muscles. Further, the system 100 may in addition or in place of responding to any physiological signal, respond to direct physical properties like temperature(s), pressure(s), muscle movement (extension or contraction), and other properties that may be suitably sensed and/or utilized by the present system including for feedback control of the system 100. For example, the present system may sense an extension of an operating (first) hemi-diaphragm (e.g., healthy normal operation and/or abnormal operation) or simply a condition indicative of a signal to extend the hemi-diaphragm, to trigger stimulation of the phrenic nerve to cause movement of the other (second) hemi-diaphragm in patients that only need unilateral pacing or to adjust pacing of the first hemi-diaphragm when it is operating abnormally (e.g., partial movement or no movement). The system 100 may similarly sense other properties that are external to the system 100, such as physiological conditions, atmospheric conditions, etc., for active diagnostics either of the system 100 (itself), of other devices and/or of other systems. For example, the present system may be utilized for diagnosing and/or observing a physiological condition wherein there is stimulation of the diaphragm but little or no muscle movement response. Naturally and as should be readily appreciated, the present system may similarly sense, through use of the expansion I/O, properties of external (e.g., external to the system 100) and internal systems for purposes of data logging the properties to record the data, such as physiological data, for later analysis. In such an embodiment, the present system may perform a continuous recording of properties, for example in one or more memories, such as the memories associated with one or more of the master and slave processors or an other memory. For example, the present system in this embodiment may sense and record diaphragm contraction including abnormalities across one or more of the hemi-diaphragms and/or percentages of dissolved oxygen in the blood as well other properties that may be sensed and thereby may be recordable.

In one embodiment, the system 100 may, based on a sensed/recorded signal from an external device, control other parameters of the present system. For example, in an anti-lock braking system, performance of the present system may be adjusted based on one or more signals indicative of an airbag deployment, road conditions, atmospheric conditions (e.g., temperature, pressure, barometer, precipitation) etc.

As should be readily appreciated, through use of the expansion I/O, the system 100 may be coupled to any device including, for example, an analog/digital converter, a microcontroller, and/or other components. In this way, operation of the system 100 may be controlled and/or synchronized to another device and/or the system 100 may control and/or synchronize another device. For example, in an embodiment of the present system, the system 100 may be coupled to another system 100 through corresponding expansion I/Os. In an embodiment wherein the systems 100 may each be diaphragmatic pacemakers, one diaphragmatic pacemaker may be coupled to another diaphragmatic pacemaker. In this embodiment, one diaphragmatic pacemaker may operate as a master while the other may operate as a slave. In another embodiment, both diaphragmatic pacemakers may operate in tandem (e.g., synchronized) or each diaphragmatic pacemaker may operate independent of the other diaphragmatic pacemaker, yet still monitor operation of the other diaphragmatic pacemaker through use of the expansion I/Os.

In one embodiment, the expansion I/O may be configured to output one or several analog and/or digital signals indicating a selected parameter or parameters of operation. The parameter(s) may for example, be provided to an external display or other medical and/or diagnostic device(s). The present system may provide through the expansion I/O a signal to an annunciator and/or alarm station. In another embodiment, the system 100 may send through the expansion I/O port, messages to a remote computer when parameters are changed, for example in a case of alarm conditions, and/or diagnostic parameters, battery voltages, trend values, or other data for statistical, diagnostics, data logging and/or backup purposes.

In one embodiment, the user I/O 180 may be simply a dial, button, etc., for setting operating parameters (e.g., number of breadths per minute) for the system 100 and/or a display to display the set parameters. In another embodiment, the user I/O 180 may be provided with a display screen that may enable a more detailed presentation of an operating state of the system 100, for example including a past state of the system 100, diagnostic states, etc. In one embodiment, the display screen and data presented thereon may enable a more complex adjustment to the system 100 by the user or another device through use of the user I/O 180.

The user I/O 180 may also be operable to produce an indication, such as an auditory (e.g., tone, beep, etc.) and/or visual indication, including an indication of a failure present in the system 100 operation. For example, in a case wherein the master processor 110 determines that a failure is present in operation of the slave processors 120, 130, the master processor 110 may initiate the failure indication through use of the user I/O 180, such as by initiating an audible tone and/or a visual signal such as a flashing visual signal. In one embodiment, different combinations of the auditory and/or visual indication may be utilized to identify different failure conditions. Iconic visualizations, such as pictorial representations of particular failure conditions may also be provided by the user I/O 180. The slave processors 120, 130 may also be coupled to the user I/O 180, typically for generating a failure indication similar to the master processor 110 as described further herein, although other housekeeping of the system (e.g., change in operating parameters, update and monitor user I/O 180, etc.) may also be supported by the slave processors 120, 130 as may be readily appreciated.

Further operation of the system 100 will be described in accordance with an illustrative operation in accordance with an embodiment of the present system. FIGS. 2A, 2B show a flow diagram 200, comprised of portions 200A, 200B, illustrating failure-free operation in accordance with an embodiment of the present system. Operation begins during act 210. In an embodiment wherein operation is not continuous, act 210 may be initiated each time that the system 100 is powered on, such as for a braking system, after a corresponding automobile is started. For a continuous operation, such as for a diaphragmatic pacemaker, the start act 210 may be initiated once following implantation of corresponding stimulating electrodes, such as phrenic nerve electrodes, and may continue thereafter endlessly, periodically, etc., until being purposefully inactivated or until catastrophic failure, such as when each of the processors 110, 120, 130 fails, rendering further operation impossible unless a second system 100 where available as described further herein.

In an application wherein diagnostics are performed periodically (e.g., every hour, every day, at an end of a processing cycle, at an end to one or more operations or another period that may or may not be predetermined), the master processor 110 and/or the slave processors 120, 130 may perform diagnostics on a provided power supply such as read a voltage of replaceable battery/batteries and/or of a line-level power supply. Icons representing percentages of battery capacity (e.g., representing 100%, 75%, 50% and 25%) may be lit, giving the patient or care giver a precise idea of what the capacity of the replaceable battery/batteries is and how long they will last. When the battery voltage is below 10% or other value, a battery icon may blink and an auditory alarm such as a buzzer may sound a warning beep periodically (e.g., every minute). From this moment on and until the battery is replaced, the voltage of the rechargeable batteries may be scanned at a faster rate than previously. In a case wherein the replaceable battery level depletes further (e.g., below 5%), the user indication may change to indicate a further depletion of power resources. For example, in one embodiment the buzzer may sound a more constant warning beep, such as after every breath. In a case wherein the replaceable battery level is below a lowest acceptable value, the master processor 110 may disable the replaceable battery by switching to an alternate power source if one is available. In this case, further indications may be provided by the user I/O 180.

A further diagnostic routine may include ensuring that a downstream system (e.g., downstream of the switches 140, 150), such as antennas in a case of an externally mounted diaphragmatic pacemaker, are properly attached (e.g., plugged in) and powered. In an embodiment, a connection to and/or operation of further systems may be determined during diagnostics. In a case of a detected failure during diagnostics, a suitable indication may be generated on the user I/O 180 and/or data related to the failure may be stored for later retrieval. In one embodiment, a check of the storage space utilized for storage of data is performed to determine that adequate storage exists prior to an attempt to store the data. In the same or another embodiment, the data stored may be date and/or time-stamped to facilitate a determination of when the data is acquired.

In one embodiment, such as when operation is discontinuous, diagnostic operations may be performed upon startup of the system (e.g., act 210 or following thereafter) to determine proper operation upon startup and/or periodically thereafter. In this embodiment, in a case of a determined fault condition, failure operation may progress similar to when a failure is detected after initiation of operation as described herein, such as operation of one of flow diagrams 300, 400 corresponding to a detected fault condition.

Following the start act 210, the master processor 110 sends a start signal to each of the slave processors 120, 130 as a command for each of the slave processors 120, 130 to initiate an operation during act 215. The operation of one of the slave processors 120, 130 may be the same, similar, or different than the operation of another of the slave processors 120, 130. In one embodiment, the operations may progress such that the processors 120, 130 operate in tandem with each other.

For example, in a diaphragmatic pacemaker application, each of the slave processors may operate to produce a series of control pulses. During fault-free operation of the slave processors 120, 130 in accordance with an embodiment of the present system, the control pulses are transmitted from the slave processors 120, 130 to corresponding RF sections 160, 170 through corresponding switches 140, 150. In response to receipt of the control pulses, the RF sections 160, 170 may produce corresponding waveforms, such as RF waveforms. In one embodiment, the RF sections 160, 170 may be preprogrammed to produce one or more selectable RF waveforms having specific operational parameters, such as frequency, pulse width, amplitude, and waveform characteristics that are selected by the received control pulses. In another embodiment, the control pulses may define the RF waveforms specifically by identifying each of the operational parameters of the RF waveforms.

The waveforms may represent stimulus pulses that are applied transdermally to implanted receivers for applying stimulus to phrenic nerves and thereby, stimulating breathing in the user. For example, the RF sections 160, 170 may be operationally coupled to antennas that are placed over respective skin areas of the user. Corresponding implanted receivers of an implanted device may be located right below the skin areas and stimulus transmission of the implanted device may be performed through the RF sections 160, 170.

In a system 100 that is an implanted diaphragmatic system, the RF sections 160, 170 may be eliminated and the phrenic nerve stimulating device may be connected directly to the output of the switches 140, 150. Additionally, for some other applications, the output signal from the processors 110, 120, 130 and/or switches 140, 150 may be sent to an external device to directly stimulate phrenic or other nerves, systems, etc., bypassing RF/antenna/receiver sections.

Although in a totally implantable pacemaker there may be no visual or auditory alarms, an operably coupled (e.g., wired, wireless, optical, etc.) secondary unit may have alarm signals, parameters or any other information transmitted to and/or from the system 100. For example, the secondary unit may be a base station, a watch, a pager, a cell phone, a wireless station connected to a computer or any device operably coupled to the system 100, for example communicating wirelessly (e.g., via RF). In one embodiment of an implanted system 100, the user (e.g., a patient and/or caregiver) may easily check the operating parameters and/or diagnostic information by reading the display of the secondary device, such as a wrist watch or a chronometer held by a neck strap. The secondary device may also operate to program the system 100, verify and/or test the system's operating parameters, etc.

In accordance with an embodiment, the operations of the slave processors 120, 130 may be independent and/or synchronous. In a diaphragmatic pacemaker application, the output from one of the slave processors 120, 130 may be different from the other of the slave processors 120, 130. For example, one side of a diaphragm that is being controlled by the present system may require a different number of pulses, different pulse widths, amplitudes, etc. as compared to the other side. However in this embodiment, the start of the breath cycle may be synchronized so that stimulation of both hemidiaphragms starts at the same time.

In another embodiment, the slave processors 120, 130 may operate as control portions of a braking system. In this embodiment, the processors may monitor braking, speed, acceleration, road conditions, etc., to suitably apply a braking action via the switches 160, 170 to different portions (e.g., different sides, front/back, and/or diagonally) of braking elements, such as brake calipers and/or rotors. Other applications would readily occur to a person of ordinary skill in the art and are included within the scope of the present system.

In a case wherein the start control signal is sent by the master processor 110 and no malfunction is detected (e.g., during acts 225, 235, 250, etc.), the master processor supervises operation of the slave processors 120, 130, such as during acts 235, 250 and also performs housekeeping tasks during act 220. Any one or more of these acts may be viewed as diagnostic in nature. Housekeeping tasks may include receiving user input from the user I/O 180 (e.g., reading an input keypad), sending updated data to the user I/O 180 (e.g., updating a display), performing diagnostics, such as system diagnostics, individual element diagnostics (e.g., slave processor, switch, I/O diagnostics, etc.), and logging results of the diagnostics and parameter data in the memory to enable future retrieval.

The slave processors 120, 130 monitor that a start control signal is received from the master processor within a determinable (e.g., from length of instruction execution of processors), predetermined, or adjustable (e.g., via the I/O 180) amount of time to ensure that the master processor 110 is operating properly during act 225. Presuming that the start control signal from the master processor 110 is received within the predetermined amount of time, the slave processors 120, 130 may each send an acknowledgement signal to the master processor 110 acknowledging receipt of the start control signal during act 230. The acknowledgement signals enable confirmation by the master processor 110 during act 235 that the slave processors 120, 130 are working properly and are initiating or continuing generation of corresponding operations, such as initiating corresponding control pulse trains related to a new breath in a diaphragmatic pacemaker application.

In response to the start control signal and following or concurrent with sending of the acknowledgement signals in an embodiment wherein one is provided, the slave processors 120, 130 each generate control signals that are received by the corresponding switches 140, 150 during act 240, such as a programmed number of pulses for each breath in a diaphragmatic pacemaker application. The master processor 110 monitors the output signal of each slave processor 120, 130 during act 245 to determine that each signal and timing are correct during act 250, for example, at the end of each signal portion (e.g., pulse train). In one embodiment wherein the control signals from the slave controllers 120, 130 have a defined end, at the end of a last signal generated, each slave processor 120, 130 sends an end signal to the master processor 110 during act 255 (e.g., indicating that stimulation related to one breath has finished). The master processor 110 receives each of the end signals and checks if the timing and the number of signal portions are correct during act 260. In a case wherein each part of the system is verified to operate properly, the operation during act 260 may return to act 215 under control of the master controller 110.

In a case wherein signals received by the master processor 110 from either or both slave processors 120, 130 are not within operating limits (e.g., frequency, amplitude, waveform, etc.) or are not present at all during one or more of acts 235, 250, 260, then one or both slave processors are not operating properly and operation may pass to a fault detection/operation (e.g., starting at act 310) as shown in FIG. 3 in accordance with an embodiment of the present system.

FIG. 3 shows a flow diagram 300 illustrating a faulty slave processor operation in accordance with an embodiment of the present system. During act 310, the master processor 110 generates a failure indication on the user I/O 180 to indicate that a failure has occurred. Details of the failure condition may also be provided as the details are discerned by the master processor 110, for example following act 320. During act 320, the master processor 110 determines whether only one of the slave processors 120, 130 is not operating properly based on the responses received by the master processor 110. In a case wherein only one of the slave processors 120, 130 is not operating properly, the master processor 110, then, disables the slave processor that is not working properly and sends a command to the other slave processor during act 330 to take up the task of also generating the signals typically produced by the disabled slave processor in a fault-free operating condition. For example, in a case wherein the slave processor 120 is deemed faulty during act 320, the slave processor 130 is commanded by the master processor 110 to generate the signals for the switch 140 that the slave processor 120 would typically produce in a case where no fault is present in the slave processor 120. In this way, the path between the slave processor 130 and the switch 140 that is typically not utilized in fault-free operation, is utilized to ensure continued operation of the system. Similarly, in a case wherein the slave processor 130 is deemed faulty during act 320, the slave processor 120 is commanded by the master processor 110 to generate the signals for the switch 150 that the slave processor 130 would typically produce in a case where no fault is present in the slave processor 130. In this way, the path between the slave processor 120 and the switch 150 that is typically not utilized in fault-free operation, is utilized to ensure continued operation of the system. Operation may continue with act 215 with the one disabled slave processor and a modified operation accounting for having one operational slave processor.

In another embodiment, the master processor 110 may take over operation for the disabled processor. In yet another embodiment, the two operational processors (the master and operational slave) may degrade into a lockstep processor operation. Other systems of accounting for a non-operational slave processor would readily occur to a person of ordinary skill in the art and are included within the present system.

In a case wherein both slave processors 120, 130 are deemed to not be operational during act 320, the master processor 110 may generate a suitable notification during act 340, such as a visual or audible notification through the user I/O 180. The master processor 110 may disable both slave processors 120, 130, for example by disabling a power source of the slave processors 120, 130, and take over operation for both slave processors 120, 130 during act 350. In this embodiment, the master processor 110 may generate the signals for the switches 140, 150 that the slave processors 120, 130 typically produce in a case where no fault is present in the slave processors 120, 130 to ensure continued operation of the system. In another embodiment, the master processor 110 may only generate signals typically produced by one of the slave processors 120, 130. For example, in one embodiment, operation of one of the slave processors 120, 130 may be deemed more critical than another of the slave processors 120, 130, and accordingly, operation of the more critical slave processor is continued at the expense of the operation portions typically supported by the less critical slave processor.

In a case wherein the master processor 110 is not operating properly or is not operating at all, the slave processors 120, 130 may detect that the proper start signal is not received from the master processor 110, or is not received within the proper time during act 225. In this case, the slave processors may continue operation as illustratively shown in FIG. 4.

FIG. 4 shows a flow diagram 400 illustrating a faulty master processor 110 operation in accordance with an embodiment of the present system. In operation, one or more of the slave processors 120, 130 may disable the master processor 110 during act 405. In one embodiment in accordance with the present system, the slave processors 120, 130 may each send a disable command that when both are received by a polling circuit, such as an AND logic circuit that may be internal to the master processor 110 or may be separately configured, keeps the master processor 110 in a reset state indefinitely until the system 100 may be serviced. In this way, a two-out-of-two voting system ensures that the master processor 110 is faulty as opposed to a failure that generates a master processor 100 disable signal that is a result of a failure of one of the slave processors 120, 130. In another embodiment, a redundant processor may be applied to ensure that a failure has occurred in the master processor 110, or either of the slave processors 120, 130 as discussed further herein. In such an embodiment, any of the processors 110, 120, 130 may in fact be comprised of one or more redundant processors for purposes of determining a failure in the master processor 110, or either or both of the slave processors 120, 130. In one embodiment, one or more of the processors 110, 120, 130 may be configured as a plurality of processors acting as lockstep processors. In this way, any one or more of the processors 110, 120, 130 may operate independently to ensure failure-free operation. In case of a determined failure, the faulty processor may disable itself or be disabled by another processor as described herein, and operation of the system may continue. As may be readily appreciated, other systems for ensuring that a detected failure of the master processor 110 is actually a failure of the master processor 110 and not a result of another failure, such as a failure of one of the slave processor 120, 130, may be readily applied in accordance with the present system.

A suitable failure indication may be generated by one or more of the slave processors 120, 130, for example indicating the failure condition during act 410. The slave processors 120, 130 may communicate with each other during act 420 to ensure that both slave processors 120, 130 are operational during act 430. In a case wherein both slave processors 120, 130 are operational, each may communicate to each other and continue to work together as if the master processor 110 where operational to ensure continued operation of the system during acts 440, 450. For example, in one embodiment, the slave processors 120, 130 may communicate together to enable operation in tandem and synchronization to enable continued operation of the system.

In another embodiment in accordance with the present system, the slave processors 120, 130 may degrade into a lockstep processor operation wherein one of the slave processors 120, 130 operates as a master processor of the lockstep processor, such as generating control signals, timing signals, etc., while the other of the slave processors 120, 130 operates as a slave processor of the lockstep processor generating signals for the switches 140, 150 to enable continued operation of the system.

When only one of the slave processors 120, 130 is determined operational during act 430, for example through use of a redundant processor, the slave processor that is not operating properly may be disabled by the other slave processor during act 460. For example, in one embodiment, the failure-free slave processor may continue generating signals for one or more of the switches 140, 150 during act 450. For example, the slave processor 120 may continue generating signals for the switch 140 and may additionally generate the signals for the switch 150 when the slave processor 130 is not operational. In this way, operation of the system may continue. In another embodiment, the operational slave processor may simply perform the tasks typically performed by that processor, while not performing the tasks performed by the inoperable slave processor. In this case, operation of the system may continue, however the operation is degraded by the loss of signals that are typically generated during fault-free operation by the currently, non-operational slave processor.

While failure operation of the system 100 has been illustratively described, further systems for ensuring failure free operation may be readily applied in accordance with the present system. As such, any of the fault systems described in FIGS. 3 and 4 may be applied as a result of failure detection, for example during a diagnostic procedure. For example, in one embodiment in accordance with the present system, failure of the master processor 110 and/or one or more of the slave processors 120, 130 may be determined as a result of a self-diagnostic process running on the determined faulty device. Any one or more of the processors 110, 120, 130 may perform periodic self-test processes to determine proper operation. In one embodiment, a self-test operation may perform a known operation to generate a known result that may be performed at a time wherein one or more of the switches 140, 150 are disabled so as not to propagate a self-test signal to a downstream system (e.g., diaphragmatic pacing system, ABS braking system, etc.). Wherein a generated output does not match an expected output, the faulty processor may disable itself without further action from any of the failure-free processors.

In another embodiment, one or more of the processors 110, 120, 130 may include a watchdog timer operation. In a case wherein the watchdog timer of a given one of the processors is not restarted during operation, the given one of the processors will be reset by the watchdog timer to avoid the given processor from propagating a failure. Similarly, a watchdog timer may be utilized to generate a failure indication in case of a catastrophic failure, such as if none of the processors 110, 120, 130 are operational.

In another embodiment wherein one or more systems 100 is utilized, each of the systems may monitor each other system through the expansion I/Os to determine if a failure has occurred. In a case wherein an additional system 100 is employed, a failure-free system 100 may operate to shutdown a faulty system 100 while taking over operations typically performed by the faulty system 100. In one embodiment, each of the systems 100 may be utilized for performing different and/or separate operations during failure free operation. In another embodiment, a first system 100 may be redundant to a second system 100, thereby the first system 100 may have no operation during failure free operation except to monitor operation of the second system 100.

In accordance with an embodiment, there may also be redundancy in the power source for one or more of the devices depicted in FIG. 1. FIG. 5 shows a redundant power supply arrangement 500 in accordance with an embodiment of the present system. In the arrangement shown, there are four individual power sources, such as four temporary power sources (e.g., batteries) B1, B2, B3, B4. In one embodiment, power sources B1, B2, and B4 may be user replaceable power sources while power source B3 may not be user replaceable, such as provided by an internal lithium ion battery.

In one embodiment, batteries B1, B2, B4 may be rechargeable batteries while battery B3 may be a lithium (non-rechargeable) battery. In this way, back-up power from battery B3 may be ensured to maintain the system working without interruption, even in a case wherein the battery B3 is not utilized for some time after setup of the system. A lithium battery is known to have an extended shelf life that may be in excess of 15 years. Further, the system may be powered from an external source of power, such as line-level power, for example during times of servicing any one or more of the batteries B1, B2, B3, B4 as desired.

Each of the slave processors 120, 130 and/or corresponding portions of the system 100 may be powered by a separate one of the power sources B1, B4. Separate power supplies for each of the power sources B1, B4, respectively PS1 and PS4, may convert the voltage of each respective power source to a fixed operational output, such as three (3) volts. The power source B2 may power other circuits, such as the master processor 110. A power supply PS2 may convert the voltage from the power source B2 to a fixed output. The power source B3 may be used as a backup to assist in continuous operation of the processors 110, 120, 130 and/or associated portions of the system 100 in case one or several other power sources are exhausted or malfunction. Similarly, power supply PS3 may convert the voltage from power source B3 to a fixed output.

In operation, a supervisory circuit S2 may track the voltage of power source B2. While the voltage is within an operating range of associated circuitry, the supervisory circuit S2 may enable the power supply PS2 to receive power from the power source B2. At the same time, S2 may disable the associated circuitry from receiving power from the power supply PS3 (e.g., the internal lithium ion battery).

When the voltage of the power source B2 is below a minimum acceptable operating range for powering the associated circuitry, the supervisory circuit S2 may disable power supply PS2 and enable power supply PS3, thereby assisting the master processor 110 and/or associated circuitry to maintain power and continue to work properly without interruption.

Similarly, supervisory circuits S1 and S3 respectively, may track the voltage of power sources B1, B4. While the voltage is within an operating range of associated circuitry, supervisory circuits S1 and S3 may enable power supplies PS1 and PS4 respectively getting power from the power sources B1, B4 and may disable switching circuits SW1 and SW2 respectively, from getting power from power supplies PS2 or PS3.

In a case wherein the voltage of one or more of power sources B1, B4 is below a minimum acceptable operating range, corresponding supervisory circuit S1 and/or S3 disables corresponding power supply PS1 and/or PS4 and enables one or more of the switching circuits SW1 and/or SW2, to assist the circuitry typically powered by one or more of the power sources B1, B4 to be powered by one of power supplies PS2 or PS3 (whichever is enabled), to enable maintaining power and proper operation without interruption. As may be readily appreciated this may provide a system that is hot-swappable wherein any one or more of the power sources B1, B2, B3, B4 may be replaced even during operation of the system 100 without interruption of the operation. The redundant power supply arrangement 500 may also provide an indication to the user I/O 180 or a portion thereof (e.g., a buzzer) to provide a user alert in case of failure of any one or more of the power sources B1, B2, B3, B4.

Of course, it is to be appreciated that in accordance with the present system, any one of the above, elements, embodiments and/or processes may be combined with one or more other elements, embodiments and/or processes. As should be clear from the discussion herein, the present system overcomes disadvantages and/or makes improvements over other systems.

Finally, the above-discussion is intended to be merely illustrative of the present system and should not be construed as limiting the appended claims to any particular embodiment or group of embodiments. Thus, while the present system has been described with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function;
e) any of the disclosed elements may be comprised of hardware portions (e.g., including discrete and integrated electronic circuitry), software portions (e.g., computer programming), and any combination thereof;
f) hardware portions may be comprised of one or both of analog and digital portions;
g) any of the disclosed devices or portions thereof may be combined together or separated into further portions unless specifically stated otherwise; and
h) no specific sequence of acts or steps is intended to be required unless specifically indicated.

1. A fault-tolerant processor device comprising:
a master processor; and
a plurality of slave processors operationally coupled to the master processor for performing a plurality of operations, each slave processor performing a unique operation, wherein the master processor is configured to:
send an initiation command to each of the plurality of slave processors to initiate each of the plurality of slave processors corresponding unique operation,
monitor each of the plurality of operations to confirm that the slave processor performing the operation is fault-free,
wherein if the processor being fault-free is not confirmed,
identify and disable the faulty one of the plurality of slave processors,
initiate fault-free slave processor the plurality of slave processors to perform the one of the plurality of operations of the disabled faulty slave processor in addition to the one of the plurality of operations of the fault-free slave processor.

2. The device of claim 1, wherein the master processor is configured to
determine if of the plurality of slave processors are fault-free, and
if not, of the plurality of slave processors performing faulty operations and all of the plurality of operations of the faulty slave processors.

3. The device of claim 1, further comprising a user input/output device operationally coupled to the master processor, wherein the master processor is configured to produce a failure indication on the user input/output device if one of the plurality of slave processors is faulty.

4. The device of claim 1, wherein each of the plurality of slave processors are configured to determine if the master processor sent the initiation command, and
if the initiation command was not sent, the plurality of slave processors are configured to
disable the master processor and perform the plurality of operations without the master processor sending the initiating command.

5. The device of claim 4, wherein the determination of if the master processor sent the initiation command is performed within a predetermined period of time.

6. The device of claim 4, wherein each of the plurality of slave processors is configured to monitor the operations of another of the plurality of slave processors if the master processor is disabled.

7. The device of claim 4, comprising a user input/output device operationally coupled to at least one of the plurality of slave processors,
wherein the least one of the plurality of slave processors is configured to produce a failure indication on the user input/output device if the master processor is faulty.

8. The device of claim 1, wherein each of the plurality of slave processors is configured to acknowledge receipt of the initiation command to the master processor.

9. The device of claim 1, wherein the master processor is configured to examine a timing of each of the plurality of operations to determine if there is fault-free operation.

10. The device of claim 1, wherein the device is arranged for driving a diaphragmatic pacemaker having left and right sides, wherein one of the plurality of operations drives the left side of the diaphragmatic pacemaker, and another one of the plurality of operations drives the right side of the diaphragmatic pacemaker.

11. A method of operating a fault-tolerant processor system having a master processor and a plurality of slave processors operationally coupled to the master processors, the method comprising acts of:
sending an initiation command from the master processor to each of the plurality of slave processors;
initiating operation by the slave processors in response to the initiation command;
each slave processor performing a unique operation of a plurality of operations during fault-free operation;
monitoring each of the plurality of operations to confirm that the slave processors are fault-free; and
if the slave processors being fault-free is not confirmed,
identifying a faulty slave processor of the plurality of slave processors,
disabling the faulty slave processor, and
initiating of a fault-free slave processor of the plurality of slave processors to perform the one of the plurality of operations of the faulty slave processor in addition to the one of the plurality of operations of the fault-free slave processor.

12. The method of claim 11, comprising acts of determining if any of the plurality of slave processors are fault-free, and
if none of the plurality of slave processors are fault-free, disabling all of the plurality of slave processors, and performing each of the plurality of operations by the master processor.

13. The method of claim 11, comprising an act of producing a failure indication if one of the plurality of slave processors is faulty.

14. The method of claim 11, comprising acts of:
determining if the master processor sent the initiation command and
if the initiation command was not sent, disabling the master processor, and initiating operation by the slave processors without interaction from the master processor.

15. The method of claim 14, wherein the act of determining if the master processor sent the initiation command comprises an act of determining if the master processor sent the initiation command within a predetermined period of time.

16. The method of claim 14, comprising an act of monitoring the operations of each of the plurality of slave processors by all of the plurality of slave processors if the master processor is disabled.

17. The method of claim 14, further comprising an act of producing a failure indication if the master processor is faulty.

18. The method of claim 14, wherein the act of monitoring each of the plurality of operations to confirm the fault-free operation comprises an act of examining a timing of each of the plurality of operations.

19. A diaphragmatic pacemaker having at least two sides, the pacemaker comprising:
a master processor; and
a plurality of slave processors operationally coupled to the master processor for performing a plurality of operations, each slave processor performing a different unique operation, wherein the master processor is configured to
send an initiation command to each of the plurality of slave processors to initiate each of the plurality of slave processors corresponding unique operation, each of at least two of the slave processors controlling a corresponding different side of the diaphragmatic pacemaker during fault-free operation,
monitor that each side of the diaphragmatic pacemaker is properly controlled, and
if any side is not properly controlled,
identify and disable a faulty one of the plurality of slave processors performing the improper control, and
initiate of a fault-free slave processor of the plurality of slave processors to control both sides of the diaphragmatic pacemaker.

20. The diaphragmatic pacemaker of claim 19, comprising a user input/output device operationally coupled to the master processor,
wherein the master processor is configured to produce a failure indication if one of the plurality of slave processors is faulty.
